(19) United States
(12) Patent Application Publication
(10) Pub. No.: US 2016/0319234 A1
(43) Pub. Date: Nov. 3, 2016

(54) CONTINUOUSLY CONTROLLED HOLLOW FIBER BIOREACTOR

(71) Applicant: L&J Biosciences Inc., Gaithersburg, MD (US)

(72) Inventors: Jeong Kuen SONG, Gaithersburg, MD (US); Eun Sung YANG, Gaithersburg, MD (US); Sookhee BANG, Gaithersburg, MD (US); Sungwoo Roger KANG, Gaithersburg, MD (US)

(21) Appl. No.: 15/143,541

(22) Filed: Apr. 30, 2016

Related U.S. Application Data
(63) Continuation-in-part of application No. PCT/US14/64420, filed on Nov. 6, 2014.
(60) Provisional application No. 61/900,920, filed on Nov. 6, 2013.

(51) Int. Cl. C12M 1/12; C12M 1/00; C12M 1/34

(52) U.S. Cl. CPC C12M 25/10; C12M 41/26; C12M 41/34; C12M 41/12; C12M 29/00; C12M 23/28

(57) ABSTRACT

Continuously Controlled Hollow fiber Bioreactor (CCHB) for the production of consistent quality cells or cell-derived products is provided. General components of the CCHB including a hollow fiber cell culture module, a new-medium chamber and a used-medium chamber, disposably attachable or detachable to the base devices such as rocking platform and circulation pumps. Quality of parameters including nutrient, oxygen, pH and temperature in the medium is optimally maintained during the production process. This ensures the controlled quality of the cell or cell-derived product throughout the process.

### BACKGROUND OF THE INVENTION

1. Field of the Invention

The present invention relates generally to a system and a method for continuously culturing and harvesting cells and cell derived products. Further, this invention relates to consistent production of high quality cells or cell-derived products via controlled culture environment and parameters.

2. General Background and State of the Art

Semipermeable hollow fibers as a cell culture system has been used over decades for the production of cell and cell-derived products, including proteins, peptides, antibodies, hormones, and vaccines. Hollow fiber type bioreactor provides several advantages. For example, it provides a large amount of surface area, which facilitates local distribution of nutrients to the cells and collection of cell waste and metabolites. In addition, cells can grow to higher density compared with other cell culture systems, mimicking in vivo environment. Hollow fiber type bioreactors can support cell densities greater than 10⁸ cells per milliliter, whereas other cell culture systems such as conventional stainless steel type bioreactors allow for cell density of less than 10⁶ cells per milliliter.

Hollow fiber culturing system provides in vivo like environment and allows growth of cells in serum free medium or medium that contains less serum than conventionally used media. Secreted cell-derived products can be concentrated by the filter-like character of the hollow fiber, which is typically 100 times higher than that can be carried out with classical bioreactors. Hollow fiber type bioreactor is typically a single-use bioreactor, which means that it is a closed and disposable system. An advantage of a single-use bioreactor is the significantly reduced cleaning and sterilization costs compared with a fixed asset stainless steel bioreactor. And such single-use bioreactor easily passes complex qualification and validation procedures. Low cross-contamination risk and increased safety of process are other advantages.

However, existing designs of hollow fiber type bioreactors have some drawbacks. For example, conventionally known hollow fiber type bioreactor apparatus does not provide for application of turbulent energy in the cell culturing process. Turbulent energy is typically required to achieve even distribution of cell and medium throughout the cell culture space. Also, existing hollow fiber bioreactors do not provide for control of various parameters for cell growth, such as temperature, pH, nutrient amount, gas, glucose consumption, cell density and cell number throughout the culture space. In addition, hollow fiber bioreactors lack active oxygen delivery capability to the culture space. The inability to control the optimum cell culturing parameters in these hollow fiber bioreactors limits the production volume of the culture to less than a hundred liters, which is useful for only small scale laboratory research purposes, and is not suitable for production at an industrial scale. Therefore, limited culture size of current hollow fiber bioreactor design is a major barrier for use of this system to mass culturing system at an industrial level.

Controlled parameter and culture environment in a bioreactor are significant for industrial production of cells or cell-derived bio-products. For example, unstable or inconsistent culture environment results in uneven distribution of healthy growing cells, forming a mixture of cell fates or undesirable, modified cell-derived bio-products causing contamination of the resultant product sample.

Therefore, what is needed in the field is a bioreactor and a method of producing cell and cell-derived products that incorporate tight and precise control of parameters of the cell culture environment even in large scale. The present invention provides an apparatus and method for readily and constantly controlling the parameters for optimum cell culture production, applicable to manufacturing biologic medical products.

### SUMMARY OF THE INVENTION

The invention provides a unique system of Continuously Controlled Hollow fiber Bioreactor (CCHB) as cell propagation system. The present invention is also directed to a process of making cell-derived products using the inventive CCHB.

The CCHB is designed for the efficient and quality production of biological or pharmaceutical materials. For example, a book sized (about 2 liter reaction vessel) CCHB can produce ˜3.5 kg of monoclonal antibody from a single batch, which is comparable to that which can be obtained from a conventional bioreactor that is about 1500 liters in scale. Therefore, CCHB system can save substantial amount of space, reagent, labor and cost. Dynamic and high rate of exchange of gas, nutrient and metabolic waste at high cell density are some of the significant features of the inventive CCHB system.

In one aspect, the present invention provides a cell culture system that has the following characteristics.
a consistently clean environment for the bioreactor, which includes sterile disposable system such as the module, tubes, sensors and accessories.
a harvesting control functionality controlled by the pore size on fibers according to desired molecular cutoff ranges.
a cell growth control functionality in which the cells are mixed well and thus cell distribution and density is controlled by rocking motion.
gentle sheering force caused by rocking motion through the fibers, ensuring safe and healthy environment for the cells.
control of oxygen supply depending on size of the culture.
control for parameters such as but not limited to temperature, media supply, gas, and pH.
a scalable system from research laboratory usage to industry scale, which may be up to thousands of liters.
medium perfusion system that constant quality of medium is provided during the process.
relatively small space requirement, which results significant space cost saving.
capability of sample concentration by TFF (Tangential Flow Filtration) system.

Some of the advantages of the inventive bioreactor system may include, without limitation, the following. A broad range of cell types such as human, animal, plant and bacterial cells may be cultured. A broad range of culture size to industrial scale (more than thousands of liters) may be processed. Both suspension and adherent cell culture may be grown and cultured. Heterologously mixed cell culture including co-culture (within for example the extra-capillary space) may be carried out in the inventive bioreactor system. Stem cell or primary cell culture where matrix and cytokines are required may be also grown and cultured. Cell culture with micro-carrier may be used in the inventive bioreactor. Monoclonal antibody may be manufactured. Recombinant protein or bio-medicine may be produced. PK/PD (Pharmacokinetic/Pharmacodynamic) determinations for in vitro toxicology may be carried out. Cytokines and growth factors may be produced. Cell cultures may be monitored real time. And, cell expansion (including suspension, adherent, primary, lymphocyte and stem cell) may be carried out using the inventive bioreactor.

In another aspect, the present invention is directed to a high throughput hollow fiber bioreactor.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 is a general schematic illustration of the Continuously Controlled Hollow fiber Bioreactor (CCHB) of the invention.

FIG. 2 is a sectional front view of a hollow fiber cell culture module on a rocking platform.

FIG. 3A is a schematic illustration of a hollow fiber cell culture module.

FIG. 3B is a front view of the hollow fiber cell culture module of 3A.

FIG. 3C is a sectional side view of the hollow fiber cell culture module of 3A.

FIG. 3D is a plan view of the hollow fiber cell culture module of 3A.

FIG. 4A is a schematic illustration of the hollow fiber culture with small pore size.

FIG. 4B is a schematic illustration of CCHB comprising small pore sized hollow fiber.

FIG. 5A is a schematic illustration of the hollow fiber culture with large pore size.

FIG. 5B is a schematic illustration of CCHB comprising large pore sized hollow fibers. Used media collection is concentrated through TFF (Tangential Flow Filtration) system.

FIG. 6A is a schematic illustration of the hollow fiber culture with microcarriers.

FIG. 6B is a schematic illustration of CCHB comprising large pore sized hollow fibers with micro-carriers.

FIGS. 7A, 7B, 7C, 7D, 7E,7F, 7G and 7H show different ways of supplying gas to the culture module depending on application.

FIG. 7A is a schematic illustration of direct aeration by gas permeable silicon tubing in the module.

FIG. 7B is a schematic illustration of direct aeration by gas sparging in a new media container.

FIG. 7C is a schematic illustration of dynamic aeration chamber following by cell culture module.

FIG. 7D is a schematic illustration of air diffusion through hollow fiber oxygenator.

FIG. 7E is a schematic illustration of air sparging chamber.

FIG. 7F is a schematic illustration of coiling of air permeable silicon tubing.

FIG. 7G is a schematic illustration of stirring media reservoir with gas exchangeable sterile filter.

FIG. 7H is a schematic illustration of media aeration chamber with gas exchangeable sterile filter.

FIGS. 8A, 8B and 8C show aseptic replacement of disposable containers of new/used media.

FIG. 8A is a schematic illustration of new media container replacement.

FIG. 8B is a schematic illustration of used media container replacement.

FIG. 8C is a schematic illustration of aseptic multi-tab connector system.

### DETAILED DESCRIPTION OF THE PREFERRED EMBODIMENTS

In the present application, “a” and “an” are used to refer to both single and a plurality of objects.

This invention is not limited to certain applications, protocols and reagents described herein and as such, may vary. The terminology used herein is for the purpose of certain embodiments only, and is not intended to limit the scope of the disclosed invention, which is defined solely by the claims.

As used herein, “cell derived product” refers to proteins including growth factors, cytokines, monoclonal antibodies, immunoglobulin products, enzymes, hormones, vaccines and fusion proteins.

As used herein, “hollow fibers” are small tube-like filters approximately 200 microns in diameter whose molecular weight cut-off can be between 10 kD and 0.2 μm. These fibers are typically sealed into a cartridge shell so that cell culture medium pumped through the end of the cartridge will flow through the inside or outside of the fiber while the cells are grown inside or outside of the fiber depending on the size of the pores or the various conditions in and surrounding the hollow fibers. These fibers then create a semi-permeable barrier of defined molecular weight cut-off (MWCO) between the compartment in which the cells are growing and the medium is flowing. Since the cells are attached to a porous support (the hollow fiber) rather than a non-porous plastic dish nutrients are delivered readily delivered to the cells. For instance, the pore size of the hollow fibers may differ depending on how the hollow fibers are to be used. For example, hollow fibers with molecular weight cut off (MWCO) ranging from 10 kD to 1000 kD or up to 0.2 μm of pore size may be used for certain purposes.

As used herein, “hollow fiber cell culture module” or the “module” means the housing which contains hollow fibers where cells are cultured and interior space of hollow fiber where media passes.

As used herein, “high throughput hollow fiber bioreactor” means a type of hollow-fiber bioreactor, which is equipped for high capacity of nutrient, gas and waste exchange to perform commercial scale cell culture. In one aspect, the amount of cell-derived product obtained from the inventive bioreactor may be a large amount relative to the size of the reaction vessel of the bioreactor, and relative to conventionally known bioreactors of similar size. The nutrient, gas and waste exchange may be carried out at a rapid rate to support the cells that produce the cell-derived product.

The outer dimensions of the bioreactor may include small scale to medium scale to large scale to mega-large scale. Small scale bioreactor may have outer dimensions in the range of about 1 cm×7 cm×10 cm (inside bioreactor reaction volume of about 30 ml). Medium scale bioreactor may have outer dimensions in the range of about 3 cm×12 cm×22 cm (inside bioreactor reaction volume of about 400 ml). Medium-large scale bioreactor may have outer dimensions in the range of about 5 cm×22 cm×35 cm (inside bioreactor reaction volume of about 2 liters). Large scale bioreactor may have outer dimensions in the range of about 10 cm×60 cm×60 cm (inside bioreactor reaction volume of about 20 liters). Mega-large scale bioreactor may have outer dimensions in the range of about 20 cm×90 cm×120 cm (inside bioreactor reaction volume of about 100 liters). The shape of the bioreactor may be varied so long as the bioreactor functions to produce cell-derived products. For instance, the shape may not be limited to a rectangular shape. Any shape may be used so long as the object stably and effectively useable. The outer dimensions of the bioreactor may be made in accordance with the appropriate setting and environment.

As used herein, a “microcarrier” is a support matrix allowing for the growth of adherent cells in bioreactors. Microcarriers are typically 125-250 micrometer spheres and their density allows them to be maintained in suspension with gentle stirring. Microcarriers can be made from a number of different materials including DEAE-dextran, glass, polystyrene plastic, acrylamide, collagen, and alginate. Microcarriers may be used to grow protein-producing or virus-generating adherent cell populations in, without limitation, large-scale commercial production of biologics (for example, proteins) and vaccines and so forth.

Continuously Controlled Hollow Fiber Bioreactor (CCHB) System

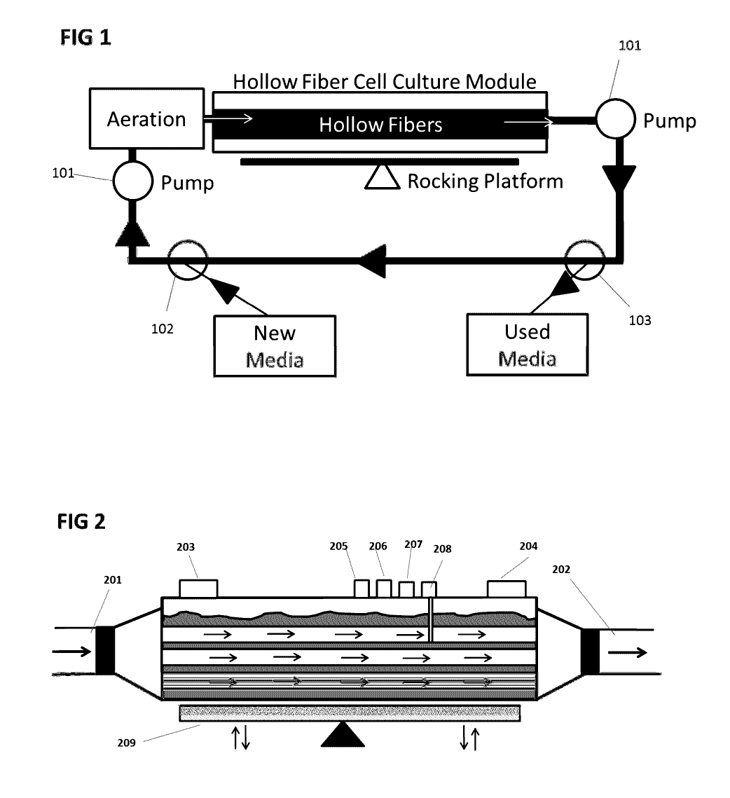

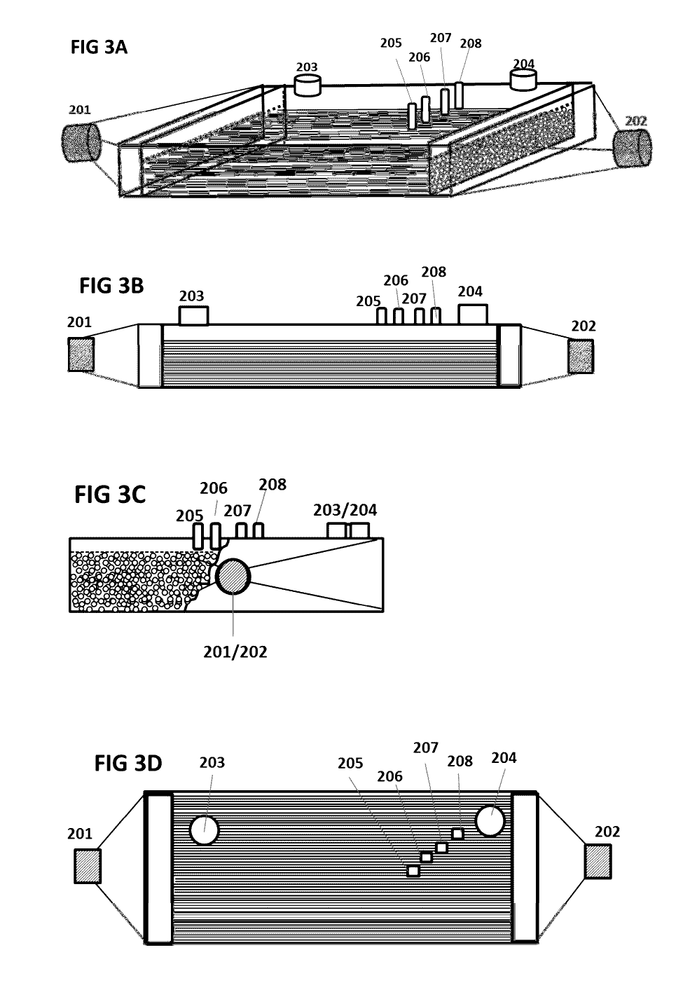

CCHB system is briefly illustrated in FIGS. 1, 2, and 3A-3D. Main components of CCHB include without limitation hollow fiber cell culture module, turbulence energy input, pump, gas supply and medium exchange. All of the considered parameters such as temperature, pH, oxygen concentration, turbulent force, flow rate, cell density and glucose consumption can be monitored and controlled.

1. Hollow Fiber Cell Culture Module

The hollow fibers are placed longitudinally in the module and sealed at each end. The hollow fibers may be made of polysulfone, polypropylene, nylon, polyester, polytetrafluoroethylene, polyethersulphone, polyethylene, polyvinylidene fluoride, cellulose acetate, mixed esters of cellulose, or a combination thereof. Also, pore size of the hollow fibers may vary depending on molecular weight cutoff (MWCO) target of the process (ranging from 10 kD to 500 kD). The apparatus housing may be plastic bag type or hard shell box type (rectangular or square) or any type or shape at all so long as the housing is able to hold the hollow fibers and the media without leakage when subjected to various culturing conditions in particular in a turbulent environment. The housing may include media inlet (201) and outlet (202) connected to the lumen of the hollow fiber cell culture module at each end (FIG. 2). The hollow fiber cell culture module houses the bundled hollow fibers. On the top of the module, aseptic inlet for cell inoculation (203) and outlet for cell harvesting (204) may be installed. In addition, disposable sensors for parameters such as, but not limited to, temperature (205), pH (206), oxygen concentration (207) may be included. Glucose/lactose concentration sensor may also be included. It is understood that the drawing is for illustrative purposes only, and not all of the sensors illustrated need to be included in the inventive bioreactor, and further, more sensors may be included as well. More aseptic sampling outlets (208) for measuring environmental parameters including cell number, density and glucose consumption may be also installed (208).

2. Turbulence Energy Input

The module may be placed on a motion plate (209), which provides turbulence energy by horizontal shaking or wave-style rocking motion. The speed and angle of the rocking motion and the horizontal shaking can be set as desired. In addition, the plate is heat-controllable. Real-time heat control during operation can be achieved by coordination between heat sensor at the module and a heat controller connected to the motion plate. In addition, whole module can be operated in a closed chamber which provides turbulence and constant temperature.

3. Pump

Peristaltic pump may be used to create directional flow through the hollow fibers. The capacity of the pump may vary depending on the size of the culture module. The pump (101) may be placed proximal to the inlet (201) of the module to pump in new media (102) (FIGS. 1 and 2). Optionally, another pump (101) can be added proximal to the outlet (202) of the module to pump out used media (103) to overcome the back pressure from the resistant force of the hollow fibers. For the small scale of CCHB, simple directional flow can be provided by flow valve and flow pump.

4. Gas Supply

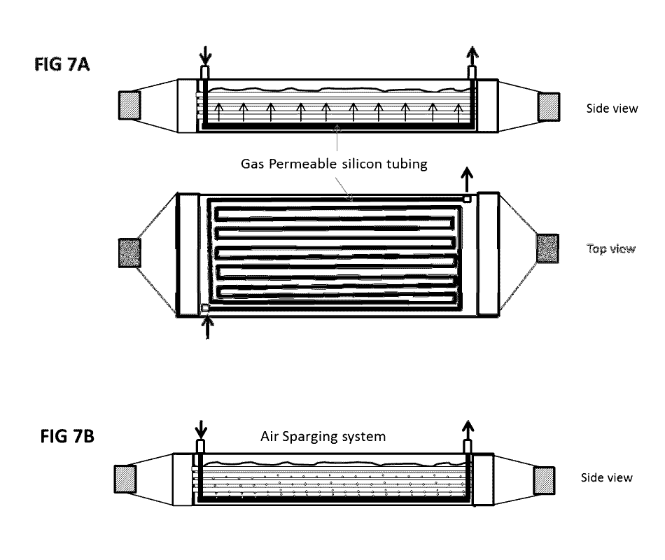

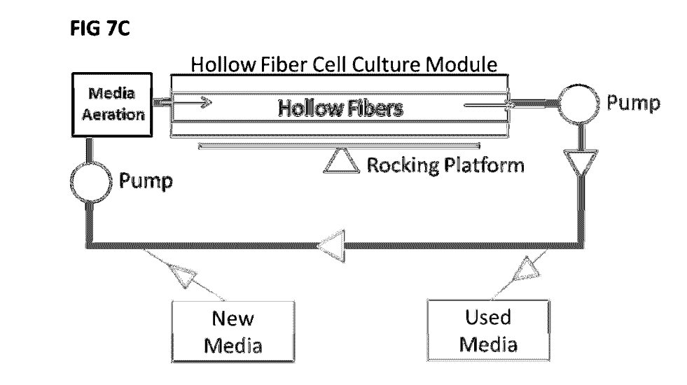

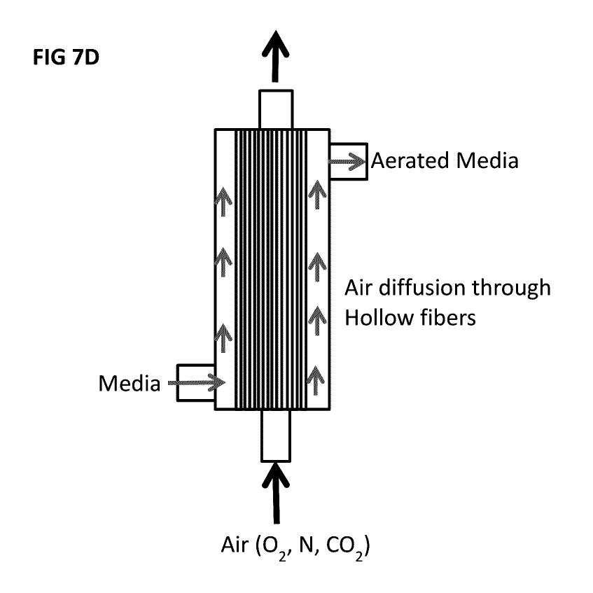

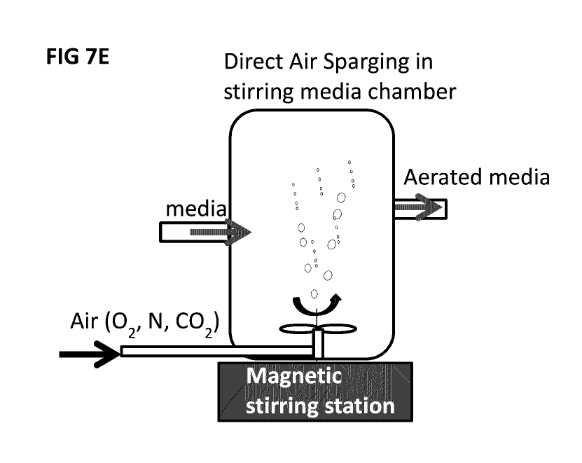

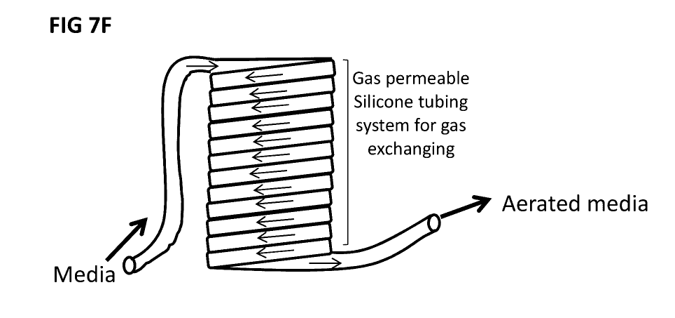

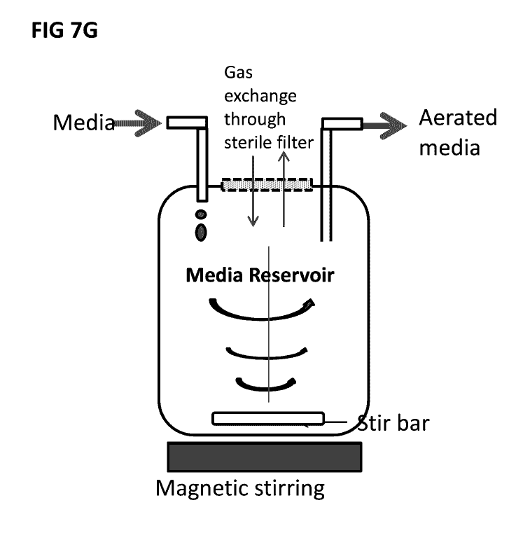

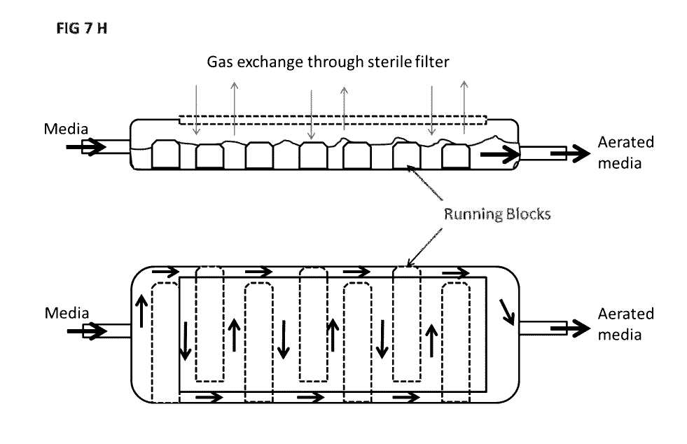

Supply of appropriate level of oxygen to the growing cells is important. Various aeration options may be chosen depending on size of the culture. As shown in FIG. 7A, aeration is provided by direct diffusion through gas permeable tubing system laid on the bottom of the cell culture module. Preferably, the gas permeable tube may be silicon tube. Alternatively, gas is sparged directly to cell culture space in the module. Gas sparger may be made of single-use materials such as including hollow fibers, metal micro/plastic sparger, or nano sparger (FIG. 7B). With the combination of turbulence motion, the aeration can be evenly distributed through the module.

Aeration can be also carried out on the circulating media (FIG. 7C). Prior to entering the module, media may be aerated in an oxygenator or air diffuser (FIG. 7D) or in a sparging chamber (FIG. 7E). Oxygenator or air diffuser can be made as single-use materials including hollow fibers. For small scale culture modules, simple diffusion through gas-permeable silicon tube may be applied (FIG. 7F). A specialized media reservoir which is equipped with stirrer with gas exchange filter can be applied as well (FIG. 7G). For this, magnetic stirring station is required. This apparatus can be made using disposable or autoclavable material. In addition, a specially designed gas exchange chamber can be used for media aeration (FIG. 7H). Passing-through media is exposed to air in a wide surface area of the chamber, where gas is freely diffused through sterile filter on the top of the chamber. Moreover, there are numbers of running blocks on the bottom of the chamber to provide longer exposure to air, while the media pass between the blocks (FIG. 7H).

5. Media Exchange

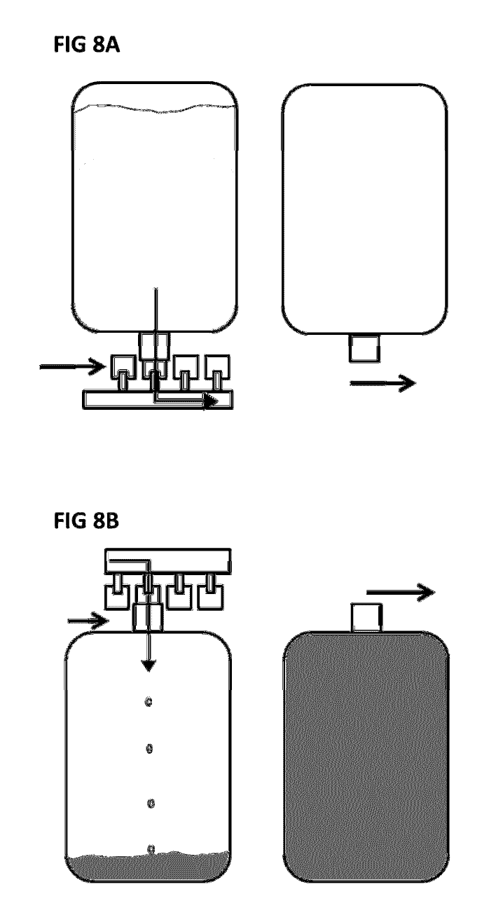

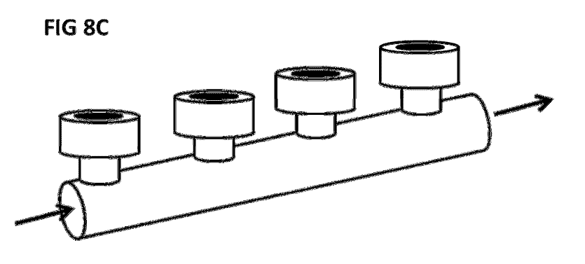

To maintain constant nutritional environment within the module, fresh media is continuously provided to the module (FIG. 1). Similar to other perfusion systems, used media may be removed from the module. New and used media containers are easily replaced without contamination (FIGS. 8A and 8B). Media containers are connected to commercially available aseptic multi-adaptors (FIG. 8C).

6. Automation Control Center

To monitor and maintain cell culturing environment in the module, sensors installed in the module are connected to the computerized control center. Programmed parameters from control center are automatically sensed and action is automatically taken in response. For example, the control center responds to a signal in the heat parameter and orders the heat plate or media reservoir to be turned on or off. pH parameter trigger causes the control to order more or less acid or base to be added to the media. Flow rate parameter signal causes the control to order the pump to be turned on or off. Turbulence parameter trigger causes the control to order the frequency of turbulence to be faster or slower. All action components can be housed together, especially in medium to large scale CCHB.

Modification of CCHB for Small Scale Production

Application of the present inventive apparatus ranges from laboratory scale to industrial manufacturing. A small version of CCHB, such as less than 30 ml module capacity, may include some modifications to reduce cost without losing performance. For example, the small CCHB does not require bulky peristaltic pump. Instead, unidirectional check flow valves can be used. In addition, since small CCHB may fit in a CO₂ incubator, the apparatus may be equipped with an oxygenator or gas-permeable silicone tubing instead of direct gas providing system described in FIGS. 7A and 7B.

Harvesting Methods

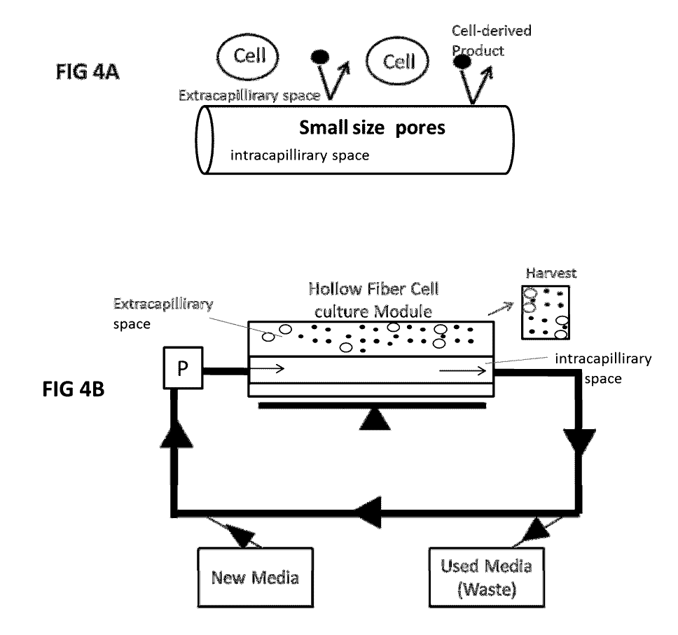

The product may be harvested in a variety of ways using CCHB. Mainly, there are two different types of harvesting methods—extra-capillary and intra-capillary harvesting procedures. First, extra-capillary harvest can be performed when the produced cell-derived product is large enough not to diffuse out into the hollow fiber intracapilliary space through the hollow fiber pores (FIG. 4A). The material to be harvested is much more concentrated (50-100 times) compared with conventional culture (FIG. 4B).

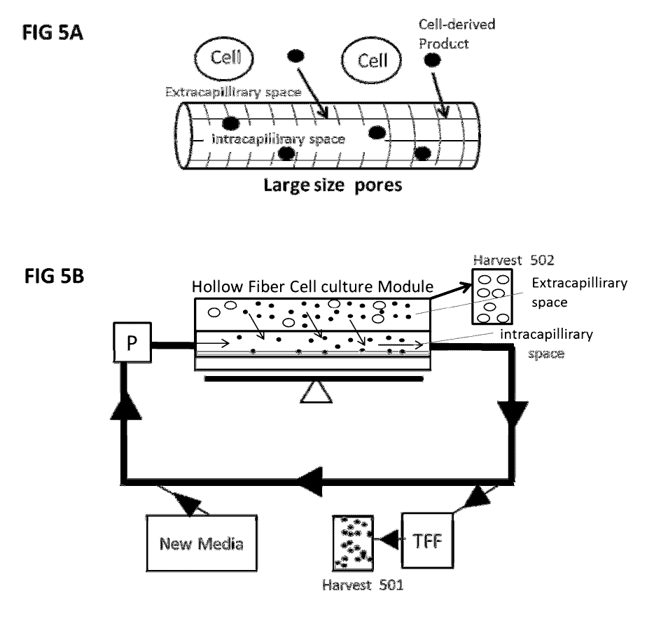

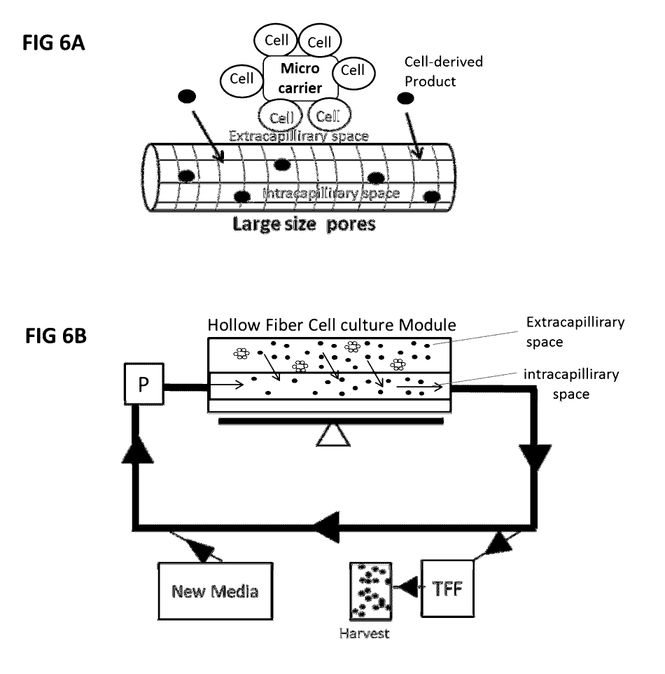

On the other hand, cell-derived product, which is small enough to diffuse out into the hollow fibers, can be collected by intra-capilliary harvest (FIG. 5A). Continuously collected sample may be concentrated by TFF (Tangential Flow Filtration) system (FIG. 5B, 501). This method also can be applied for cell expansion system by harvesting cells from extra-capilliary space (FIG. 5B, 502). Further, micro-carriers for adherent cells can be introduced into the extra-capillary space (FIG. 6A). Cell-derived product may be harvested from cell-free intra-capillary space (FIG. 6B). This will be further discussed in following section.

Dimensions of Hollow Fiber

Various pore sizes of hollow fibers can be used depending on their specific purposes. Pore sizes of MWCO ranges of 10 kD, 30 kD, 50 kD, 100 kD, 300 kD, 500 kD, 750 kD, 0.1 μm and 0.2 μm can be applied. A large pore size typically have MWCO larger than 500 kD hollow fibers allow for more efficient and faster exchange of gas, nutrient and waste. The diameter of hollow fibers is another factor to be considered. Providing hollow fibers with small diameters that typically have pore size MWCO smaller than 100 kD allows for more of the hollow fibers to be packed into the module, which results in large overall surface area but more longitudinal resistance force. In contrast, large diameter provides reduced surface area but less resistant force.

Adhesive Cell Culture Application

The inventive CCHB provides an advantage of being able to culture adherent cells, which is not readily provided for in conventional hollow-fiber systems. Many cell types such as cancer cells, primary cells, stem cells and many other tissue originated cells have an adherent characteristic. Their growth is limited by total surface area. Therefore, it is difficult to scale up for large culture. This is the major barrier to culturing adherent cells in industrial production. The inventive CCHB system may be designed to overcome this obstacle because suitably large surface area is generated from the large number of hollow fibers that may be used in the module. Further, maximal surface area per culture volume can be achieved by introducing micro-carriers and supporting matrices allowing for the growth of adherent cells in the extra-capillary space within the module (FIGS. 6A and 6B). As a result, cells grow both on the micro-carriers and on the hollow fibers with high density, enabling efficient production of cell-derived materials.

Parameter Control in CCHB System

Oxygen level control may be achieved by increase/decrease of aeration and increase/decrease of flow rate. pH control is achieved by acid/base supply. Temperature control is achieved by turning on/off of heating/motion plate. Turbulence control is achieved by increase/decrease of rocking/shaking/motion of the motion plate. Flow rate control is achieved by increase/decrease of pump flow. Glucose level control is achieved by increase/decrease of new media input. Cell density control is achieved by harvesting cells from the module. Cell viability monitoring is achieved by periodic sampling followed by viable cell counting.

Advantages of the Inventive System

In one aspect, the invention is directed to a box type configuration of the chamber that holds the hollow fibers. The box shape of the inventive apparatus is believed to be advantageous in producing cell products because no cell accumulation “dead spots” are formed as the box is shaken or is made turbulent. This is compared with a cylindrically shaped apparatus, for example, which forms “dead spots” near the fulcrum area and other areas where cells tend to gather due to minimal perturbation in the area. Such accumulation of cells would significantly reduce the effectiveness of the expression of the various cellular products. The box may be shaped such that it is a container with a substantially flat base and sides, typically square or rectangular and having a lid. The corner edges, in particular the bottom corners, may be rounded if desired. The box type configuration of the chamber may be made of any stiff substance such as glass, acrylic, hard plastic, and so forth, and is preferably not pliable.

In another aspect, an advantage of the inventive system of over the state of art includes using hosing material that would continuously flow fresh media through the hollow fibers as opposed to for example, using a reservoir system, which would cause toxins and unwanted waste products to accumulate in the reservoir, thus shortening the useful lifetime of the media as well as the culture apparatus.

In another aspect, an advantage of the inventive apparatus over the state of art is the “scalability” of the inventive apparatus. The inventive apparatus is modular in structure, in that each module can be joined one to another, in parallel or in serial, using the hosing system.

In another aspect, an advantage of the inventive apparatus over the state of art is that the rocking motion of the inventive apparatus causes even distribution of the cells, whereas any rocking motion in the state of art is designed to merely mix the media.

Those skilled in the art will recognize, or be able to ascertain using no more than routine experimentation, many equivalents to the specific embodiments of the invention specifically described herein.

1. A bioreactor comprising:
a. hollow fiber cell culture module,
b. temperature controlled motion platform,
c. circulation pump,
d. disposable medium chambers comprising fresh medium chamber and used medium chamber,
e. gas nutrient supply, and
f. harvesting unit.

2. The bioreactor of claim 1, wherein the hollow fiber cell culture module comprises an enclosed chamber for holding cells, media and hollow fiber.

3. The bioreactor of claim 2, wherein the hollow fiber cell culture module comprises
i. a housing, which comprises an enclosed chamber, having first and second ends defining a longitudinal axis of hollow fiber through the housing, wherein the hollow fiber has interior and exterior surfaces disposed within the housing,
ii. at least two ports in the housing defining fluid flow path through the interior of the fibers, wherein two spaces are securely separated, wherein
inner capillary space comprises at least one inner side lumen of the hollow fiber, and
extra capillary space comprises a space between the inner side of the enclosed chamber and outer side of the hollow fiber,
iii. at least two openings on the module for introduction and harvesting of cells and cell derived products, and
iv. ports for sensing pH, oxygen and temperature.

4. The bioreactor of claim 1, wherein the hollow fiber is semipermeable.

5. The bioreactor of claim 3, wherein the hollow fiber is composed of a material selected from polysulfone, polypropylene, nylon, polyester, polytetrafluoroethylene, polyethersulphone, polyethylene, polyvinylidene fluoride, cellulose acetate and mixed esters of cellulose.

6.-7. (canceled)

8. The bioreactor of claim 3, wherein at least one hollow fiber has a pore size no larger than 0.2 μm.

9. The bioreactor of claim 3, wherein the hollow fiber cell culture module contains a port for pH sensor.

10. The bioreactor of claim 3, wherein the hollow fiber cell culture module contains a port for oxygen sensor.

11. The bioreactor of claim 2, wherein the hollow fiber cell culture module holds at least 10⁸ cells.

12. The bioreactor of claim 2, wherein the cells are adhesive or non-adhesive mammalian cells.

13.-14. (canceled)

15. The bioreactor of claim 2, wherein the cells are adhesive or non-adhesive stem cells.

16. The bioreactor of claim 13, wherein the adhesive cells grow on a microcarrier in the extra-capillary space.

17. The bioreactor of claim 2, wherein the cells are bacterial cells.

18. The bioreactor of claim 1, wherein the gas nutrient is oxygen, carbon dioxide, nitrogen or air.

19.-25. (canceled)

26. The bioreactor of claim 1, which holds a volume of less than about 50 ml.

27. The bioreactor of claim 25, which fits inside a general type CO₂ incubator.

28. The bioreactor of claim 27, having dimensions of about 470 mm×640 mm×480 mm.

29. The bioreactor of claim 26, which does not require heating system from the plate.

30. The bioreactor of claim 30, which is shaped as a box.

31. A method of culturing cells, comprising using the bioreactor of claim 1.
